(19) United States
(12) Patent Application Publication
(10) Pub. No.: US 2026/0133656 A1
(43) Pub. Date: May 14, 2026

(54) CLOUD-BASED USER PROFILE SYSTEM FOR ADAPTIVE GUI MODIFICATION

(71) Applicant: Clemson University, Clemson, SC (US)

(72) Inventor: Julian Brinkley, Clemson, SC (US)

(73) Assignee: Clemson University, Clemson, SC (US)

(21) Appl. No.: 19/371,246

(22) Filed: Oct. 28, 2025

Related U.S. Application Data
(60) Provisional application No. 63/717,926, filed on Nov. 8, 2024.

(51) Int. Cl. G06F 3/041; A61B 3/024; A61B 5/11; A61B 5/16; B60W 50/14; G06F 3/0488; G06F 21/32; G06T 11/00

(52) U.S. Cl. CPC G06F 3/0418; A61B 3/024; A61B 5/1124; A61B 5/16; G06F 3/0488; G06F 21/32; G06T 11/10; B60W 50/14; G06T 2200/24

(57) ABSTRACT

A computer system for providing adaptive graphical user interface modifications for users with impairments is provided, comprising: a server configured to store user profiles containing accessibility parameters; a calibration computer device in communication with the server and configured to display an indicator representing a requested touch area on a touch screen, receive a plurality of touch points from a user attempting to contact the requested touch area, calculate coordinate modification values based on differences between the requested touch area and the plurality of touch points, and store the coordinate modification values in a user profile on the server; and a remote computer device having a display and configured to identify the user, retrieve the user profile from the server, and modify touch input coordinates according to the coordinate modification values stored in the user profile.

### RELATED APPLICATIONS

This application claims priority to U.S. Provisional Patent Application No. 63/717,926 filed Nov. 17, 2024, which is hereby incorporated by reference in its entirety.

### BACKGROUND OF THE INVENTION

### 1) Field of Invention

This system is directed to a cloud-based user profile system that enables remote storage and retrieval of personalized interface modifications for graphical user interfaces to assist users with visual or motor skill impairments.

### 2) Description of the Related Art

Graphical User Interfaces (GUIs) have become increasingly prevalent across numerous industries due to rapid digital transformation. The widespread integration of technology into daily life has created a growing demand for intuitive, efficient, and visually appealing interfaces. Well-designed GUIs can simplify complex processes, making software applications and devices more accessible to a broader range of users. Businesses are increasingly focused on user-centric design, as seamless interactions lead to greater customer satisfaction and retention. Additionally, GUI design has become a differentiator between competitors, as the interface is central to how users perceive and interact with various goods and services.

However, current GUI technologies present substantial challenges for users with visual impairments and motor skill impairments. GUIs rely heavily on visual elements such as icons, buttons, and layout designs, which can be difficult or impossible to navigate for visually impaired users. While these users may depend on assistive technologies like screen readers or voice commands, many applications are not optimized for such tools. Inconsistent labeling of visual elements, poor color contrast, or lack of keyboard navigation support can make it even more difficult for impaired users to interact with digital platforms.

Similarly, users with motor skill impairments face difficulties with touchscreen interfaces that require precise finger movements and accurate touch inputs on small target areas.

Users with physical impairments, such as tremors, limited fine motor control, or reduced dexterity, may experience difficulty accurately targeting digital button actuation areas on touch-sensitive input devices. When such users attempt to interact with a touchscreen interface, their actual point of physical contact may be systematically displaced from the intended digital button location due to involuntary hand movements, reduced precision in finger placement, or limitations in motor coordination. This spatial offset between the user's actual touch point and the digital button's defined actuation area can result in failed button presses, unintended activation of adjacent interface elements, or complete inability to access certain functions of the device.

The misalignment between intended and actual touch locations may be particularly pronounced for users with conditions affecting motor control, where consistent directional bias in touch placement can occur. For instance, a user may consistently touch points that are offset in a particular direction from their intended target, creating a predictable pattern of spatial displacement. Additionally, users with limited range of motion or joint mobility may find it challenging to reach certain areas of a touchscreen interface, forcing them to attempt interactions from suboptimal angles or positions that further compromise touch accuracy. These physical limitations can make standard touchscreen interfaces largely inaccessible, as the rigid nature of digital button boundaries does not accommodate the natural variation in touch precision that may accompany various physical impairments.

These accessibility challenges are particularly pronounced in modern self-service technologies such as touchscreen kiosks for point-of-sale systems and emerging autonomous vehicle interfaces. Self-service kiosks have revolutionized retail and service industries by offering faster, more efficient transaction methods, but their touchscreen-based interfaces often lack tactile feedback and screen reader compatibility. Similarly, the emergence of self-driving vehicles represents a significant evolution in personal mobility, but their Human-Machine Interfaces predominantly focus on visual indicators and conventional touchscreens, which may pose problems for users with disabilities. The shared-use nature of these technologies, where users may encounter different interface designs across various service providers, compounds these accessibility challenges.

Therefore, it is an object of the present system to provide a cloud-based user profile system that enables personalized interface modifications to be stored remotely and retrieved by various computer systems for dynamic GUI adaptation to assist users uses these device that have visual or motor skill impairments.

It is another object of the present system to provide touch input calibration and coordinate translation capabilities that compensate for motor skill impairments by mapping actual touch points to intended interface elements.

It is another object of the present system to provide display modification capabilities that adapt visual elements including color schemes, contrast levels, and text sizing to accommodate various forms of visual impairments including color blindness.

### BRIEF SUMMARY OF THE INVENTION

The above objectives are accomplished by providing a computer system for providing adaptive graphical user interface modifications for users with impairments. The computer system comprises a server configured to store user profiles containing accessibility parameters, a calibration computer device in communication with the server and configured to display an indicator representing a requested touch area on a touch screen, receive a plurality of touch points from a user attempting to contact the requested touch area, calculate coordinate modification values based on differences between the requested touch area and the plurality of touch points, and store the coordinate modification values in a user profile on the server, and a remote computer device having a display and configured to identify the user, retrieve the user profile from the server, and modify touch input coordinates according to the coordinate modification values stored in the user profile.

The calibration computer device may apply weighted averaging to the plurality of touch points when calculating the coordinate modification values. The calibration computer device may assign greater weight to touch points that demonstrate consistency and reduced weight to touch points that show deviation from an established pattern. The calibration computer device may disregard outlier touch points that fall outside acceptable deviation parameters when calculating the coordinate modification values. The remote computer device may be a self-service kiosk comprising a receipt printer and a bill acceptor. The self-service kiosk may identify the user through biometric authentication comprising facial recognition or fingerprint identification. The user profile may contain display modification parameters for accommodating visual impairments, and the remote computer device may modify display characteristics according to the display modification parameters.

The system may include a computerized device for adapting autonomous vehicle interfaces for users with disabilities. The computerized device comprises processing circuitry configured to detect user identification information through at least one of biometric authentication, card reading technology, or electronic chip communication, retrieve a user profile from a remote server based on the user identification information where the user profile contains coordinate translation parameters and display modification settings determined during a calibration process, apply the coordinate translation parameters to translate actual touch coordinates to intended interface activation coordinates, and implement the display modification settings to adapt visual elements of the autonomous vehicle interface, and a memory coupled to the processing circuitry and configured to store the retrieved user profile during vehicle operation.

The card reading technology may comprise at least one of near field communication (NFC) readers, radio frequency (RF) identification systems, chip card readers, or magnetic stripe card readers. The display modification settings may comprise color palette adjustments for accommodating color vision deficiencies including deuteranopia protanopia or tritanopia. The color palette adjustments may avoid red and green color combinations for users with deuteranopia protanopia and substitute with blues and yellows that provide visual contrast. The processing circuitry may record interaction data during vehicle operation and transmit the interaction data to the remote server for user profile refinement. The interaction data may comprise button presses, touch coordinates, and interface errors detected during user interaction with the autonomous vehicle interface.

The system may provide cloud-based accessibility adaptations across multiple interface platforms. The computer system comprises a server configured to maintain user profiles containing accessibility parameters including coordinate translation values and visual display modifications, a rehabilitation system in communication with the server and configured to conduct accessibility assessments and generate detailed accessibility profiles, and a plurality of interface devices including kiosks and autonomous vehicles where each interface device is configured to authenticate user identity through at least one identification method, retrieve a corresponding user profile from the server, apply coordinate translation to modify touch input coordinates based on the coordinate translation values, and implement visual display modifications based on the visual display modifications stored in the user profile.

The rehabilitation system may conduct clinical-grade assessment protocols including fine motor control evaluations, visual field assessments, and cognitive processing capability measurements. The rehabilitation system may generate the coordinate translation values based on systematic evaluations of motor skill capabilities and limitations identified during the clinical-grade assessment protocols. The at least one identification method may comprise biometric authentication including facial recognition systems and fingerprint identification systems. Each interface device may authenticate user identity through card reading technologies comprising near field communication readers, radio frequency identification systems, and chip card readers. The visual display modifications may comprise color palette adjustments that avoid red and green color combinations for users with deuteranopia protanopia and avoid blue and yellow color combinations for users with tritanopia. The visual display modifications may comprise high contrast adjustments that create visual separation between text and background elements and between interactive components and surrounding visual context.

### BRIEF DESCRIPTION OF THE SEVERAL VIEWS OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 is a system diagram of aspects of the cloud-based user profile system;

FIGS. 2 and 3 are system diagrams of aspects of the cloud-based user profile system;

FIG. 4 is a flowchart of aspects of the cloud-based user profile system;

FIG. 5 is a system diagram of aspects of the cloud-based user profile system; and,

FIG. 6 is a system diagram of aspects of the cloud-based user profile system.

While each of the drawing figures depicts a particular embodiment for purposes of depicting a clear example, other embodiments may omit, add to, reorder, and/or modify any of the elements shown in the drawing figures. For purposes of depicting clear examples, one or more figures may be described with reference to one or more other figures, but using the particular arrangement depicted in the one or more other figures is not required in other embodiments. The drawings and schematic representations are intended to support the understanding of the invention. These may not be to scale and are not intended to limit the invention to any particular layout, connectivity, or architectural implementation. Correspondence between drawing elements and described components is provided for illustrative purposes and should not be interpreted to limit the claim scope.

### DETAILED DESCRIPTION OF THE INVENTION

In the following description, for the purposes of explanation, numerous specific details are set forth in order to provide a thorough understanding of the present disclosure. It will be apparent, however, that the present disclosure may be practiced without these specific details. In other instances, well-known structures and devices are shown in block diagram form in order to avoid unnecessarily obscuring the present disclosure. Modifiers such as “first” and “second” may be used to differentiate elements, but the modifiers do not necessarily indicate any particular order.

With reference to the drawings, the invention will now be described in more detail.

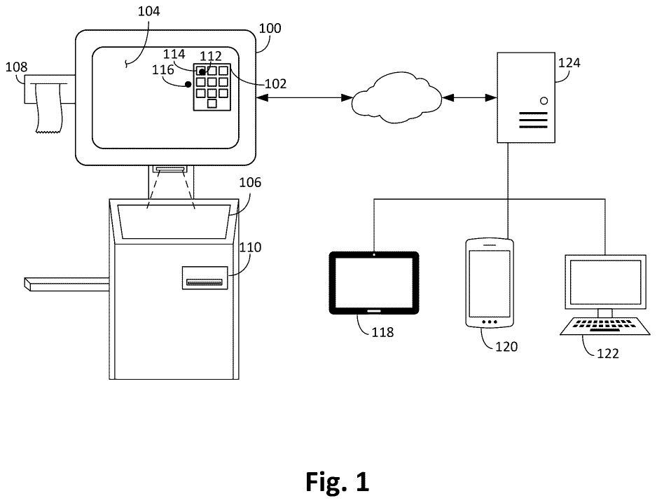

Referring to FIG. 1, a touch screen system 100 includes a keypad 102 and a touch screen display 104. The touch screen system 100 can form part of a kiosk 106 that incorporates various integrated components for user interaction and transaction processing. The kiosk 106 includes a receipt printer 108 and a bill acceptor and ejector 110 to facilitate complete transaction workflows. In one embodiment, the kiosk 106 includes a scanning area where goods can be scanned, which can include a scale for weighing goods.

The touch screen system 100 operates by detecting user interactions through touch input detection. When a user contacts the touch screen system 100, the system detects a touch screen point 112 that corresponds to an intended virtual button 114. However, users with motor skill impairments face challenges when the actual contact occurs at a user touch point 116 that falls outside the intended virtual button 114 area. This misalignment between the intended virtual button 114 and the actual user touch point 116 creates accessibility barriers for users with motor impairments.

The touch screen technology can utilize capacitive touchscreens that rely on electrical properties of the human body and use indium tin oxide coating. In this configuration, the touch screen system 100 detects changes in capacitance when a user's body, which conducts electricity, contacts the screen surface. Alternatively, the touch screen technology can utilize resistive touchscreens that work by physically pressing two conductive and resistive layers together. In resistive touchscreen implementations, the touch screen system 100 detects contact when pressure causes the conductive and resistive layers to make physical contact.

The system architecture includes networked components that enable distributed processing and data storage. A tablet 118, a smart phone 120, and a computer 122 connect through network communications to a server 124. The server 124 facilitates data exchange between the various system components and stores user profile information for accessibility adaptations.

The virtual button 114 area can be mathematically defined using coordinate parameters. The virtual button 114 has an area represented by the relationship A=(X_{w}−Xₜ)(Yₕ−Yₜ), where Xₜ and Yₜ represent the top left corner coordinates of the virtual button 114, and X_{w} and Yₕ define the width and height boundaries. The touch screen system 100 registers a successful button activation when the touch screen point 112 satisfies the conditions Xₜ≤X₁≤X_{w} and Yₜ≤Y₁≤Yₕ, where X₁ and Y₁ represent the actual touch coordinates detected by the touch screen system 100.

The system addresses motor skill impairments through a coordinate translation mechanism that modifies initial contact point coordinates to align with intended touch areas. When a user with motor impairments attempts to activate the virtual button 114 but contacts the touch screen at coordinates outside the button area, the system applies mathematical transformations to translate the actual touch coordinates to the intended button coordinates. The coordinate translation utilizes the mathematical relationships Xₑ=Xᵢ+Xᵥand Yₑ=Yᵢ+Yᵥ, where Xₑ and Yₑ represent the effective contact point coordinates, Xᵢand Yᵢrepresent the initial contact point coordinates detected by the touch screen, and Xᵥand Yᵥ represent the coordinate modification values that can be positive or negative depending on the direction of the required adjustment.

A calibration process determines the coordinate modification values Xᵥ and Yᵥ through systematic measurement of user touch patterns. During calibration, the system presents visual indicators that instruct the user to touch specific button areas on the touch screen display. The calibration system records multiple touch attempts when the user contacts areas outside the intended button boundaries. In one embodiment, the system captures a first initial point Xᵢ₁ and Yᵢ₁ during a first touch attempt, records a second touch as Xᵢ₂ and Yᵢ₂ during a second attempt, and captures a third touch as Xᵢ₃ and Yᵢ₃ during a third attempt. The calibration process continues until the recorded touch points demonstrate consistency within a predetermined grouping threshold that indicates the user contacts the touch screen within a repeatable touch area.

The calibration computer device processes the collected touch point data using statistical analysis techniques to determine accurate coordinate modification values. In one configuration, the calibration computer device calculates coordinate modifications by averaging the multiple touch points Xᵢ₁, Yᵢ₁, Xᵢ₂, Yᵢ₂, and Xᵢ₃, Yᵢ₃ to establish the Xᵥ and Yᵥ values. The calibration computer device can apply weighted averaging techniques where touch points that fall within tighter groupings receive heavier weighting in the calculation, while touch points that demonstrate greater deviation receive reduced weighting influence on the final coordinate modification values.

In one example, the calibration computer device includes outlier filtering capabilities that disregard touch points that fall outside acceptable deviation parameters. The outlier filtering process identifies touch attempts that demonstrate excessive variation from the established touch pattern and excludes these outlier touch points from the coordinate modification calculations. This outlier filtering enhances the accuracy of the coordinate translation by preventing erratic touch attempts from skewing the calculated Xᵥ and Yᵥ values.

The system accommodates various user interaction methods including users who utilize alternative contact techniques due to motor impairments. In one embodiment, the system supports users who use a knuckle instead of a fingertip to press designated areas on the touch screen. When a user employs knuckle contact instead of fingertip contact, the touch detection typically registers at coordinates offset from the intended button area due to the different contact geometry and pressure distribution. The coordinate translation mechanism compensates for this offset by applying the calculated Xᵥ and Yᵥ values to translate the knuckle contact coordinates to the intended button activation coordinates.

Once the calibration process determines the coordinate modification values, the system stores the Xᵥ and Yᵥ parameters in a user profile that can be accessed by various touch screen systems. The user profile contains the personalized coordinate translation parameters that enable consistent accessibility adaptations across different touch screen interfaces. When the user subsequently interacts with touch screen systems, the coordinate translation mechanism retrieves the stored Xᵥ and Yᵥ values and applies the mathematical transformations to translate actual touch coordinates to intended activation coordinates, thereby enabling effective touch screen interaction despite motor skill impairments.

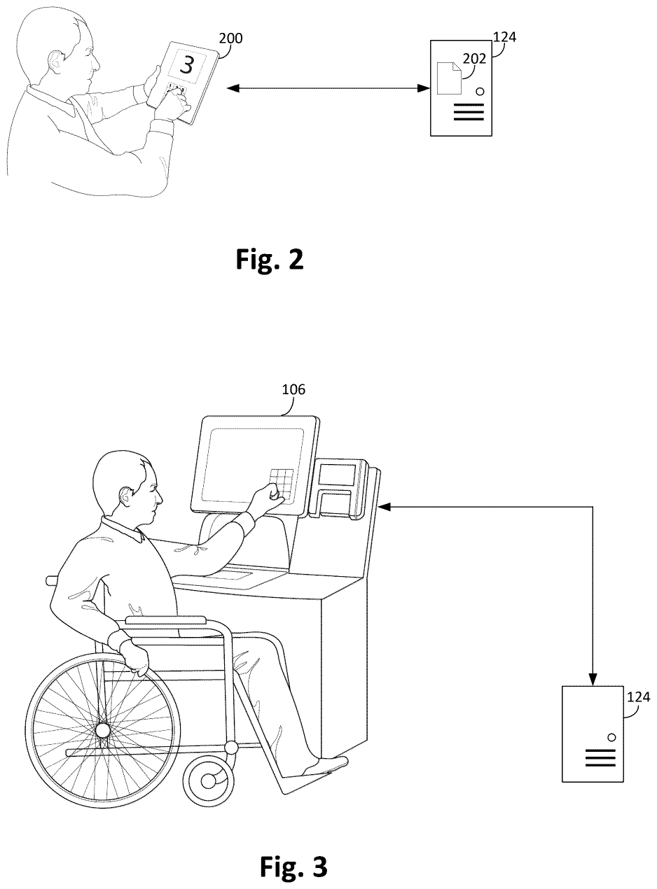

Referring to FIG. 2, a computer device 200 displays an indicator 202 that guides users through the calibration process for establishing personalized touch coordinate modifications. The computer device 200 presents the indicator 202 as a visual representation of where the user should contact the touch screen surface during calibration procedures. The indicator 202 provides clear visual guidance that enables the calibration system to establish baseline measurements for comparing intended touch areas with actual user contact points.

The computer device 200 operates by presenting the indicator 202 and monitoring user touch responses to determine coordinate offset patterns. When the computer device 200 displays the indicator 202 representing a specific button area, the system records the actual touch coordinates detected by the touch screen sensors. The computer device 200 compares the intended touch area defined by the indicator 202 with the actual touch point coordinates to calculate the difference between the requested touch area and the user's actual contact location.

The calibration process implemented by the computer device 200 captures multiple touch attempts to establish reliable coordinate modification parameters. The computer device 200 displays the indicator 202 for a requested touch area and receives a plurality of touch points from the user during repeated calibration attempts. The computer device 200 calculates the difference between the requested touch area indicated by the indicator 202 and each individual touch point, then creates coordinate modification values based on statistical analysis of the collected touch data.

In one embodiment, the computer device 200 applies weighted averaging techniques to the multiple touch point measurements. The computer device 200 assigns greater weight to touch points that demonstrate consistency and reduced weight to touch points that show significant deviation from the established pattern. The computer device 200 can implement outlier detection algorithms that disregard touch points falling outside acceptable deviation parameters, thereby improving the accuracy of the coordinate modification calculations.

Referring to FIG. 3, the kiosk 106 incorporates user identification systems that enable retrieval of personalized user profiles from the server 124. The kiosk 106 implements multiple identification methods to authenticate users and access their stored accessibility profiles. The identification systems enable the kiosk 106 to retrieve the appropriate coordinate translation parameters and display modifications for each individual user.

The kiosk 106 includes biometric authentication capabilities that identify users through biological characteristics. In one configuration, the kiosk 106 incorporates facial recognition systems that capture and analyze facial features to identify users and retrieve their corresponding profiles from the server 124. The facial recognition system compares captured facial data with stored biometric templates to authenticate user identity and access personalized accessibility settings.

In another embodiment, the kiosk 106 includes fingerprint identification systems that authenticate users through fingerprint pattern analysis. The fingerprint identification system captures fingerprint data when users contact designated sensor areas on the kiosk 106. The fingerprint identification system compares the captured fingerprint patterns with stored fingerprint templates in the user profiles maintained on the server 124 to verify user identity and retrieve appropriate accessibility modifications.

The kiosk 106 incorporates card reading technologies that enable user identification through various card-based authentication methods. In one configuration, the kiosk 106 includes near field communication (NFC) readers that detect and communicate with NFC-enabled cards or mobile devices. The NFC reader establishes communication with NFC-enabled identification cards to retrieve user identification information and access corresponding user profiles stored on the server 124.

The kiosk 106 can include radio frequency (RF) identification systems that communicate with RF-enabled cards or tags carried by users. The RF identification system detects RF signals transmitted by user identification cards and extracts user identification data to retrieve personalized accessibility profiles from the server 124. The RF identification system operates across various frequency ranges to accommodate different RF card technologies and communication protocols.

In another embodiment, the kiosk 106 includes chip card readers that interface with smart cards containing embedded microprocessors or memory chips. The chip card reader establishes electrical contact with the smart card to read stored user identification data and authentication credentials. The chip card reader communicates with the server 124 to retrieve user profiles based on the identification information extracted from the smart card.

The kiosk 106 can incorporate magnetic stripe card readers that extract user identification information from cards with magnetic stripe encoding. The magnetic stripe card reader scans the magnetic stripe data when users swipe their identification cards through the reader mechanism. The magnetic stripe card reader processes the encoded data to identify users and retrieve their accessibility profiles from the server 124.

Once the kiosk 106 identifies a user through any of the implemented identification methods, the system retrieves the corresponding user profile from the server 124. The retrieved user profile contains the coordinate translation parameters Xᵥ and Yᵥ determined during the calibration process, along with display modification settings tailored to the user's specific accessibility requirements. The kiosk 106 applies the coordinate translation mechanism to modify touch input coordinates and implements display adaptations to enable effective interaction for users with motor skill impairments or visual limitations.

The system incorporates display modification capabilities that adapt visual elements to accommodate various forms of visual impairments. The display modifications address specific types of color vision deficiencies through targeted color palette adjustments and alternative visual presentation methods. The display modification system analyzes user visual requirements during the calibration process and stores appropriate display parameters in the user profile for consistent application across different interfaces.

For users with Deuteranopia Protanopia, which affects red-green color perception, the display modification system implements specific color palette adjustments that enhance visual accessibility. The display modifications utilize blues, yellows, and other contrasting colors that remain distinguishable for individuals with red-green color blindness. The system avoids red and green color combinations that create visual confusion for users with Deuteranopia Protanopia and substitutes these problematic color pairings with blue and yellow alternatives that provide clear visual contrast.

The display modification system addresses tritanopia, which affects blue-yellow color perception, through alternative color palette implementations. For users with blue-yellow color blindness, the display modifications avoid blue and yellow color combinations that create visual accessibility barriers. The system substitutes problematic blue and yellow color pairings with shades of red, green, or grays that create sufficient visual contrast for users with tritanopia. The color substitution process maintains the functional purpose of the original color coding while providing accessible visual presentation.

In one embodiment, the display modification system implements Color Universal Design (CUD) principles that accommodate multiple forms of color vision deficiency simultaneously. The CUD color palette includes specific combinations of blue, purple, yellow, and orange that provide accessible visual presentation for users with various types of color blindness. The CUD implementation ensures that visual information remains accessible across different color vision capabilities without requiring separate color schemes for different types of visual impairments.

The display modification system incorporates high contrast adjustments that enhance visual accessibility for users with various visual limitations. The contrast modifications create distinct visual separation between text and background elements, as well as between interactive components such as buttons and links and their surrounding visual context. The high contrast implementation utilizes dark text on light backgrounds or light text on dark backgrounds to maximize visual distinction and readability.

The system includes texture and pattern integration capabilities that provide visual information through non-color-dependent methods. The display modifications incorporate textures and patterns in areas where color distinction would normally convey information, such as in graphs, charts, or maps. The texture and pattern system creates visual differentiation through geometric patterns, line styles, or surface textures that remain distinguishable regardless of color perception capabilities.

In one configuration, the display modification system implements text label additions that supplement or replace color-based information presentation. The text labeling system adds descriptive text to areas where color coding would typically convey information to users. The text labels provide explicit information about data categories, status indicators, or functional elements that would otherwise rely on color distinction for user comprehension.

The display modification system incorporates tooltip functionality that provides additional textual information when users interact with visual elements. The tooltips appear when users hover over or contact specific interface elements, providing descriptive text that explains the function or meaning of visual components. The tooltip system ensures that users can access complete information about interface elements without relying on color-based visual cues.

In another embodiment, the display modification system includes legend integration that provides textual explanations for visual coding systems. The legends accompany graphs, charts, or other visual presentations that utilize color coding or pattern coding to convey information. The legend system creates comprehensive textual descriptions that explain the meaning of different visual elements, enabling users to interpret visual information through text-based descriptions rather than color perception.

The display modification system stores all visual adaptation parameters in the user profile maintained on the server. The stored display modifications include color palette selections, contrast settings, texture and pattern preferences, and text labeling configurations. When users access different interfaces through the kiosk or other connected systems, the display modification system retrieves the stored visual adaptation parameters and applies the appropriate modifications to ensure consistent accessibility across different platforms and devices.

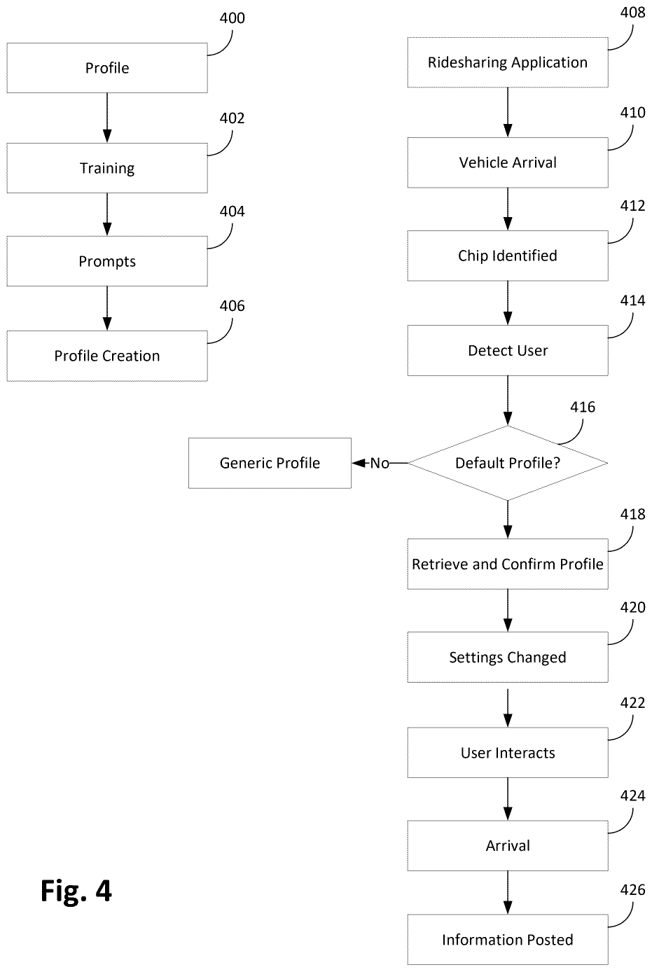

Referring to FIG. 4, the system implements a comprehensive autonomous vehicle profile creation and application process that enables personalized accessibility adaptations for users with disabilities. The process begins with a user profile 400 that serves as the foundation for collecting and storing individual accessibility requirements and preferences. The user profile 400 contains personalized parameters that accommodate specific visual impairments, motor skill limitations, and interface preferences for autonomous vehicle interactions.

The system initiates an initial training 402 phase that guides users through a systematic process for establishing their personalized accessibility parameters. The initial training 402 presents users with a structured sequence of interactions designed to capture their specific accessibility requirements and interface preferences. During the initial training 402, the system evaluates user responses to various interface elements and interaction methods to determine optimal accessibility configurations.

The system implements a provide prompts 404 stage that presents users with specific questions and interaction scenarios to assess their accessibility needs. The provide prompts 404 phase includes visual assessments, touch interaction evaluations, and audio preference determinations that enable the system to understand individual user requirements. The provide prompts 404 process captures data about color vision capabilities, contrast preferences, touch accuracy patterns, and audio output preferences through systematic user interactions.

Following the assessment phase, the system executes a create user profile 406 process that compiles the collected accessibility data into a comprehensive user profile. The create user profile 406 stage processes the information gathered during the provide prompts 404 phase and generates personalized accessibility parameters including coordinate translation values, display modification settings, and audio interface preferences. The create user profile 406 process stores the compiled accessibility parameters on the server 124 for retrieval by autonomous vehicle systems.

The autonomous vehicle interaction sequence begins with a ridesharing application 408 that enables users to request transportation services from autonomous vehicle providers. The ridesharing application 408 interfaces with autonomous vehicle dispatch systems to coordinate vehicle assignments and communicate user accessibility requirements. The ridesharing application 408 transmits user identification information to the autonomous vehicle system to enable profile retrieval and accessibility adaptations.

The process continues with vehicle arrival 410 where the autonomous vehicle reaches the user's designated pickup location. During vehicle arrival 410, the autonomous vehicle system activates user detection protocols and prepares to implement accessibility adaptations based on the user's stored profile. The vehicle arrival 410 stage initiates communication protocols between the autonomous vehicle and the user's identification devices to establish user identity and profile access.

The system implements chip information retrieved 412 functionality that enables automatic user identification through various electronic identification methods. The chip information retrieved 412 process detects and communicates with NFC-enabled devices, RFID tags, or other electronic identification systems carried by users. The chip information retrieved 412 stage extracts user identification data from electronic devices to facilitate automatic profile retrieval without requiring manual authentication procedures.

The autonomous vehicle system executes detect user and retrieve user profile 414 operations that combine user identification with profile access from the server 124. The detect user and retrieve user profile 414 process authenticates user identity through the chip information retrieved 412 data and establishes communication with the server 124 to access stored accessibility parameters. The detect user and retrieve user profile 414 functionality ensures that the appropriate accessibility adaptations are available for implementation in the autonomous vehicle interface.

The system includes user confirms profile use 416 verification that enables users to approve the application of their stored accessibility settings. The user confirms profile use 416 stage presents users with options to accept their default accessibility profile or select alternative configuration options. The user confirms profile use 416 process provides users with control over which accessibility adaptations are applied during their autonomous vehicle interaction.

Following user confirmation, the system implements user profile retrieved 418 operations that download the complete accessibility parameter set from the server 124. The user profile retrieved 418 process transfers coordinate translation values, display modification settings, audio preferences, and interface adaptation parameters to the autonomous vehicle system. The user profile retrieved 418 stage ensures that all personalized accessibility data is available for implementation in the vehicle's human-machine interface.

The autonomous vehicle system applies user interface attributes modified 420 adaptations that implement the retrieved accessibility parameters in the vehicle's interface systems. The user interface attributes modified 420 process adjusts display colors, contrast levels, button sizes, and coordinate translation parameters according to the user's stored preferences. The user interface attributes modified 420 stage transforms the standard autonomous vehicle interface into a personalized accessibility-adapted interface that accommodates the user's specific requirements.

The adapted interface enables user interacts with human machine interface 422 functionality that allows users to control autonomous vehicle systems through their personalized interface adaptations. The user interacts with human machine interface 422 process applies coordinate translation for touch inputs, implements visual adaptations for display elements, and provides audio feedback according to user preferences. The user interacts with human machine interface 422 stage enables effective autonomous vehicle control despite motor skill impairments or visual limitations.

The autonomous vehicle system includes provide information about destination 424 capabilities that deliver location-specific information through accessible presentation methods. The provide information about destination 424 functionality vocalizes personalized information about restaurants and locations based on the user profile parameters stored on the server 124. The provide information about destination 424 process adapts information delivery methods to accommodate visual impairments by providing detailed audio descriptions of destination characteristics, accessibility features, and relevant location information.

The system concludes with operational information stored 426 data collection that captures user interaction patterns and system performance metrics. The operational information stored 426 process records button presses, touch coordinates, interface errors, and user feedback during the autonomous vehicle interaction. The operational information stored 426 functionality transmits the collected interaction data to the server 124 for analysis and potential refinement of the user's accessibility profile parameters, enabling continuous improvement of the personalized accessibility adaptations.

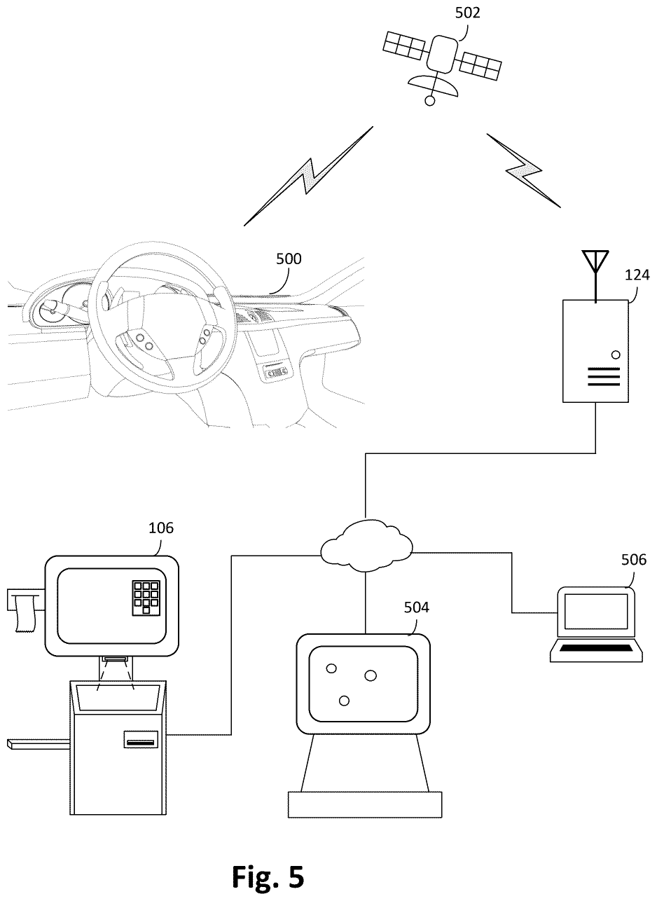

Referring to FIG. 5, the system architecture incorporates distributed network communications that enable comprehensive accessibility adaptations across multiple platforms and environments. An autonomous vehicle 500 establishes wireless communication with a satellite 502 to facilitate real-time data exchange and navigation services. The autonomous vehicle 500 communicates with the server 124 through network connections that enable retrieval of user profiles and accessibility parameters for personalized interface adaptations.

The autonomous vehicle 500 can implements wireless communications such as cellular and satellite-based communication systems that provide connectivity for profile synchronization and data updates. The satellite 502 enables the autonomous vehicle 500 to maintain communication with the server 124 even in areas with limited terrestrial network coverage. The satellite 502 communication link ensures that user accessibility profiles remain accessible and can be updated in real-time based on user interactions and system performance data.

The distributed network architecture includes the kiosk 106 as an integrated component that shares accessibility data with the server 124. The kiosk 106 contributes user interaction data and calibration information to the centralized profile database maintained on the server 124. This integration enables consistent accessibility adaptations across different interaction platforms, allowing users to experience similar interface modifications whether interacting with the kiosk 106, the autonomous vehicle 500 or other interface input-outout devices.

A rehabilitation system 504 connects to the server 124 to provide specialized accessibility assessment and training capabilities. The rehabilitation system 504 incorporates advanced calibration tools and therapeutic assessment protocols that enhance the accuracy of user profile development. The rehabilitation system 504 can conduct comprehensive evaluations of motor skill capabilities, visual acuity measurements, and cognitive assessment procedures that contribute detailed accessibility parameters to user profiles stored on the server 124.

The rehabilitation system 504 implements clinical-grade assessment protocols that capture precise measurements of user capabilities and limitations. The rehabilitation system 504 conducts systematic evaluations of fine motor control, gross motor function, visual field assessments, and cognitive processing capabilities. The rehabilitation system 504 generates detailed accessibility profiles that include specific coordinate translation parameters, optimal display configurations, and recommended interaction methods based on clinical assessment results.

A remote access terminal 506 provides distributed access to the user profile system and enables remote monitoring and potentially adjustment of accessibility parameters. The remote access terminal 506 connects to the server 124 through secure network communications that allow authorized personnel to review user profiles, analyze interaction data, and implement profile modifications if needed. The remote access terminal 506 enables healthcare providers, accessibility specialists, and technical support personnel to remotely assist users with profile optimization and troubleshooting.

The remote access terminal 506 incorporates secure authentication protocols that protect user privacy while enabling authorized access to accessibility profiles. The remote access terminal 506 implements role-based access controls that limit profile modification capabilities based on user authorization levels. Healthcare providers can access clinical assessment data through the remote access terminal 506, while technical support personnel can modify interface parameters and coordinate translation settings.

The system architecture enables broader applications of the personalized user experience technology to various autonomous systems and robots beyond transportation applications. The fundamental concept of cloud-based accessibility profiles can be applied to humanoid robots, automated service systems, and interactive kiosks across multiple industries. The coordinate translation mechanisms and display adaptation technologies developed for the autonomous vehicle 500 can be implemented in hospital robots, retail automation systems, and assistive technology devices.

In one embodiment, the system supports hospital environments where patients interact with various humanoid robots that provide medical assistance, information delivery, and therapeutic support. The humanoid robots retrieve patient accessibility profiles from the server 124 and implement personalized interface adaptations that accommodate visual impairments, motor skill limitations, and cognitive processing requirements. The humanoid robots apply coordinate translation for touch interactions, implement visual display modifications, and provide audio feedback according to patient-specific accessibility parameters.

The system incorporates gaze input technology as an advanced component of the coordinate modification system that enhances accessibility for users with severe motor impairments. The gaze input system tracks eye movement patterns and translates gaze coordinates to interface activation commands. The gaze input technology captures eye position data and correlates gaze direction with intended interface elements to enable hands-free interaction capabilities.

The gaze input system integrates with the existing coordinate translation mechanisms to provide comprehensive accessibility adaptations. The gaze input technology measures the difference between initial gaze point coordinates and intended interface element coordinates, then stores these gaze-based coordinate modifications in the user profile alongside touch-based translation parameters. The gaze input system enables users with limited motor function to interact with touch screen interfaces through eye movement control while maintaining the personalized accessibility adaptations established through the calibration process.

The coordinate modification system incorporates gaze input data as part of the difference calculation between initial contact points and intended touch areas. The gaze input measurements contribute additional coordinate translation parameters that account for eye-hand coordination variations and gaze-based interaction preferences. The system stores gaze input coordinate modifications in the user profile using similar mathematical relationships as touch-based translations, where gaze coordinate adjustments are applied to translate detected gaze positions to intended interface activation coordinates.

The distributed network architecture enables seamless integration of gaze input technology across multiple platforms including the autonomous vehicle 500, the kiosk 106, and connected robotic systems. The gaze input coordinate modifications stored in user profiles on the server 124 can be retrieved and applied by any connected system that incorporates gaze tracking capabilities. This integration ensures that users can experience consistent gaze-based interface adaptations across different autonomous systems and robotic platforms, providing comprehensive accessibility support for individuals with complex motor impairments.

According to one embodiment, the processes, techniques and functionality described herein are implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the processes, techniques and functionality, or may include one or more general purpose hardware processors configured, adapted and programmed to perform the processes, techniques and functionality pursuant to program instructions, such as computer readable instructions, in firmware, memory, other storage, or a combination. Such special-purpose computing devices may also combine custom hard-wired logic, ASICs, or FPGAs with custom programming to accomplish the processes, techniques and functionality. The special-purpose computing devices may be desktop computer systems, portable computer systems, handheld devices, networking devices or any other device that incorporates hard-wired and/or program logic to implement the processes, techniques and functionality.

One or more different inventions may be described in the present application. Further, for one or more of the invention(s) described herein, numerous embodiments may be described in this patent application, and are presented for illustrative purposes only. The embodiments described are not intended to be limiting in any sense. One or more of the invention(s) may be widely applicable to numerous embodiments, as is readily apparent from the disclosure. These embodiments are described in sufficient detail to enable those skilled in the art to practice one or more of the invention(s), and it is to be understood that other embodiments may be utilized and that structural, logical, software, electrical and other changes may be made without departing from the scope of the one or more of the invention(s). Accordingly, those skilled in the art will recognize that the one or more of the invention(s) may be practiced with various modifications and alterations. Particular features of one or more of the invention(s) may be described with reference to one or more particular embodiments or figures that form a part of the present disclosure, and in which are shown, by way of illustration, specific embodiments of one or more of the invention(s). It should be understood, however, that such features are not limited to usage in the one or more particular embodiments or figures with reference to which they are described. The present disclosure is neither a literal description of all embodiments of one or more of the invention(s) nor a listing of features of one or more of the invention(s) that must be present in all embodiments.

Headings of sections provided in this patent application and the title of this patent application are for convenience only, and are not to be taken as limiting the disclosure in any way.

It is understood that the above descriptions and illustrations are intended to be illustrative and not restrictive. It is to be understood that changes and variations may be made without departing from the spirit or scope of the following claims. Other embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventor did not consider such subject matter to be part of the disclosed inventive subject matter.

What is claimed is:

1. A computer system for providing adaptive graphical user interface modifications for users with impairments, the computer system comprising:
a server configured to store user profiles containing accessibility parameters;
a calibration computer device in communication with the server and configured to:
display an indicator representing a requested touch area on a touch screen,
receive a plurality of touch points from a user attempting to contact the requested touch area,
calculate coordinate modification values based on differences between the requested touch area and the plurality of touch points, and
store the coordinate modification values in a user profile on the server; and
a remote computer device having a display and configured to:
identify the user,
retrieve the user profile from the server, and
modify touch input coordinates according to the coordinate modification values stored in the user profile.

2. The computer system of claim 1, wherein the calibration computer device is further configured to apply weighted averaging to the plurality of touch points when calculating the coordinate modification values.

3. The computer system of claim 2, wherein the calibration computer device assigns greater weight to touch points that demonstrate consistency and reduced weight to touch points that show significant deviation from an established pattern.

4. The computer system of claim 1, wherein the calibration computer device is further configured to disregard outlier touch points that fall outside acceptable deviation parameters when calculating the coordinate modification values.

5. The computer system of claim 1, wherein the remote computer device is a self-service kiosk comprising a receipt printer and a bill acceptor.

6. The computer system of claim 5, wherein the self-service kiosk is further configured to identify the user through biometric authentication comprising facial recognition or fingerprint identification.

7. The computer system of claim 1, wherein the user profile further contains display modification parameters for accommodating visual impairments, and the remote computer device is further configured to modify display characteristics according to the display modification parameters.

8. A computerized device for adapting autonomous vehicle interfaces for users with disabilities, the computerized device comprising:
processing circuitry configured to:
detect user identification information through at least one of biometric authentication, card reading technology, or electronic chip communication,
retrieve a user profile from a remote server based on the user identification information, the user profile containing coordinate translation parameters and display modification settings determined during a calibration process,
apply the coordinate translation parameters to translate actual touch coordinates to intended interface activation coordinates, and
implement the display modification settings to adapt visual elements of the autonomous vehicle interface; and
a memory coupled to the processing circuitry and configured to store the retrieved user profile during vehicle operation.

9. The computerized device of claim 8, wherein the card reading technology comprises at least one of near field communication (NFC) readers, radio frequency (RF) identification systems, chip card readers, or magnetic stripe card readers.

10. The computerized device of claim 8, wherein the display modification settings comprise color palette adjustments for accommodating color vision deficiencies including deuteranopia protanopia or tritanopia.

11. The computerized device of claim 10, wherein the color palette adjustments avoid red and green color combinations for users with deuteranopia protanopia and substitute with blues and yellows that provide visual contrast.

12. The computerized device of claim 8, wherein the processing circuitry is further configured to record interaction data during vehicle operation and transmit the interaction data to the remote server for user profile refinement.

13. The computerized device of claim 12, wherein the interaction data comprises button presses, touch coordinates, and interface errors detected during user interaction with the autonomous vehicle interface.

14. A computer system for providing cloud-based accessibility adaptations across multiple interface platforms, the computer system comprising:
a server configured to maintain user profiles containing accessibility parameters including coordinate translation values and visual display modifications;
a rehabilitation system in communication with the server and configured to conduct accessibility assessments and generate detailed accessibility profiles; and
a plurality of interface devices including kiosks and autonomous vehicles, each interface device configured to:
authenticate user identity through at least one identification method,
retrieve a corresponding user profile from the server,
apply coordinate translation to modify touch input coordinates based on the coordinate translation values, and
implement visual display modifications based on the visual display modifications stored in the user profile.

15. The computer system of claim 14, wherein the rehabilitation system is further configured to conduct clinical-grade assessment protocols including fine motor control evaluations, visual field assessments, and cognitive processing capability measurements.

16. The computer system of claim 15, wherein the rehabilitation system generates the coordinate translation values based on systematic evaluations of motor skill capabilities and limitations identified during the clinical-grade assessment protocols.

17. The computer system of claim 14, wherein the at least one identification method comprises biometric authentication including facial recognition systems and fingerprint identification systems.

18. The computer system of claim 17, wherein each interface device is further configured to authenticate user identity through card reading technologies comprising near field communication readers, radio frequency identification systems, and chip card readers.

19. The computer system of claim 14, wherein the visual display modifications comprise color palette adjustments that avoid red and green color combinations for users with deuteranopia protanopia and avoid blue and yellow color combinations for users with tritanopia.

20. The computer system of claim 19, wherein the visual display modifications further comprise high contrast adjustments that create distinct visual separation between text and background elements and between interactive components and surrounding visual context.
